(19) United States
(12) Patent Application Publication
(10) Pub. No.: US 2016/0213453 A1
(43) Pub. Date: Jul. 28, 2016

(54) METHOD FOR PRODUCING A DENTAL PROSTHESIS

(71) Applicant: Ingo Heinzel, Bonn (DE)

(72) Inventor: Ingo Heinzel, Bonn (DE)

(21) Appl. No.: 14/917,504

(22) PCT Filed: Sep. 13, 2013
(86) PCT No.: PCT/EP2013/069017

§ 371 Date: Mar. 8, 2016

(51) Int. Cl. A61C 13/00; A61C 13/08; A61B 1/04; A61B 1/24; A61B 1/00

(52) U.S. Cl. CPC A61C 13/0004; A61B 1/24; A61B 1/00009; A61B 1/04; A61C 13/0022; A61C 13/081

(57) ABSTRACT

A method for producing a dental prosthesis including the following steps. Production of an image of the front view of the patient's face in which the patient's mouth is open and the set of the patient's teeth being replaced is visible. Superimposition of the patient's teeth in the image of the patient's face by the digital photograph of a selected set of teeth by image processing software. Adaptation of the size and/or shape of the selected set of teeth in the photograph of the patient's mouth and/or face by the image processing software. Transmission of the obtained size and/or shape of the selected set of teeth to a CAD/CAM system. Selection in the CAD/CAM system of a three-dimensional set of teeth that corresponds to the selected set of teeth. Adaptation of the three-dimensional set of teeth in the CAD/CAM system based on the size and/or shape of the selected set of teeth determined in the photograph. Milling of the dental prosthesis out of a block of material based on the size and/or shape of the adapted three-dimensional set of teeth determined in the CAD/CAM system. A dental prosthesis can also be produced using such a method.

### BACKGROUND OF THE INVENTION

1. Field of the Invention

This invention relates to a method for producing a dental prosthesis.

2. Discussion of Related Art

Some methods are basically known. For example, the publication German Patent Reference DE 102 03 664 A1 discloses a method for producing a dental prosthesis using a CAD/CAM system. In this method, a photograph is taken of the sound state of the teeth and the dental prosthesis is subsequently designed so that it corresponds to the shape at the time of the sound state.

The obtained design data for the dental prosthesis are sent to a milling device as processing commands. A block of material is subjected to milling work in order to produce the dental prosthesis. This method requires one to have a photograph of the teeth in the sound state. It is disadvantageously not possible to produce the dental prosthesis without a photograph of the teeth in the sound state.

PCT Publication WO 2009/105618 A1 discloses a method for producing a dental prosthesis in which first, a digital photograph is taken of the patient's teeth. Then the patient selects a dental prosthesis. In a software program, the patient's existing teeth are replaced in the photograph with the teeth that the patient has selected.

The patient can then view the state with the new teeth. If the patient does not like them, then he or she selects a different dental prosthesis, which in turn replaces the old teeth in the software program. These steps of selecting and comparing are carried out until the patient has found the desired shape of the dental prosthesis.

After the desired shape of the dental prosthesis has been found, the data obtained are sent to the manufacturer of the dental prosthesis. Disadvantageously, the manufacturer is only given the data from the photograph, which makes the conversion into a three-dimensional model for a CAD/CAM system relatively imprecise.

One object of this invention is to provide a method for producing a dental prosthesis that does not have disadvantages of the prior art and in particular enables an optimum adaptation of the dental prosthesis to the patient's wishes.

### SUMMARY OF THE INVENTION

The above object and others are attained with a method for producing a dental prosthesis and with a dental prosthesis according to the other claimed features.

One method according to this invention includes the following steps. Producing an image of the front view of the patient's face in which the patient's mouth is open and the set of the patient's teeth being replaced is visible. Superimposing of the patient's teeth in an image of the patient's face by the digital photograph of a selected set of teeth by image processing software. Adapting the size and/or shape of the selected set of teeth in the photograph of the patient's mouth and/or face by the image processing software. Transmitting the obtained size and/or shape of the selected set of teeth to a CAD/CAM system. Selecting the CAD/CAM system of a three-dimensional set of teeth that corresponds to the selected set of teeth. Adapting the three-dimensional set of teeth in the CAD/CAM system based on the size and/or shape of the selected set of teeth determined in the photograph. Milling the dental prosthesis out of a block of material based on the size and/or shape of the adapted three-dimensional set of teeth determined in the CAD/CAM system.

In the context of the invention, the term “set of teeth” can include only one single tooth, but can also include two, three, or more teeth. The set of teeth can also include whole rows of teeth of the upper or lower jaw.

The term “dental prosthesis” can mean the final dental prosthesis that is inserted into the mouth of the patient in the end, but in the context of the present application, “dental prosthesis” can also merely refer to a model or temporary appliance, so-called wax-up or mock-up models, which is inserted into the patient's mouth for testing purposes, for example, and can be removed again.

The method according to this invention has one advantage, among other things, of also enabling the production of temporary appliances so that it is possible, for example, to immediately test the seating of the dental prosthesis without having to simultaneously produce the final dental prosthesis. The finally determined data such as the size and/or shape of the adapted three-dimensional set of teeth can be used for various models and temporary appliances without absolutely having to carry out a new adaptation, which achieves a significant time savings and cost reduction.

The image of the patient's face can basically be produced using any image-generating method. The image can, for example, be a digital two-dimensional photograph, a film, a drawing, and/or a three-dimensional photograph, which can be produced, for example, using a facial scanner.

The superimposition of the patient's teeth by the photograph of the selected set of teeth is carried out by a suitable conventional image processing software that is known from the prior art. According to this invention, the selected set of teeth is adapted to the patient's mouth and/or face so that the selected set of teeth optimally matches the patient's mouth and/or face.

It is possible to take into account medical aspects in the process. In addition, aesthetic observations can be made that make it possible to optimize the shape and/or size. The patient can immediately see what the selected set of teeth would look like in his or her mouth and can express his or her wishes.

It is thus possible, for example, for each individual tooth in the photograph to be positioned and changed with regard to its shape and/or size until it optimally fits into the patient's mouth or the image of the patient's mouth. This can be carried out tooth by tooth until the set of teeth has been completely adapted.

If unsatisfied, the patient can select another set of teeth. It is thus advantageously possible to optimally take into account the patient's wishes. It is also advantageously possible to make optimal adaptations to the size and/or shape of the set of teeth with regard to medical and/or aesthetic aspects.

The resulting size and/or shape of the set of teeth and the associated data are recognized by the image processing software and stored if need be. According to this invention, these data are sent to the CAD/CAM system. The CAD/CAM system is a software that is known from the prior art that is used for computer-aided design and production of a dental prosthesis. The dental technician can use such a CAD/CAM system to change the size and/or shape of individual teeth or of the set of teeth.

It is thus possible, for example, to determine and measure the width of each individual tooth in the image processing software. The measured width can be input into the CAD/CAM system so that the optimally adapted width can also be used in the CAD/CAM system. This procedure can be carried out for each tooth until the entire set of teeth has been completely adapted.

The three-dimensional image of the selected set of teeth is stored or kept in electronic form in the CAD/CAM system. It is thus advantageously possible to transfer the data that is ascertained by the image processing software to the three-dimensional electronic model in the CAD/CAM system. This achieves an optimum transfer of the size and/or shape determined in the image processing software into the CAD/CAM system.

The three-dimensional set of teeth that corresponds to the photographed set of teeth is stored in the CAD/CAM system. The adaptation of the set of teeth in the CAD/CAM system can occur automatically, but it can also be performed manually by the dental technician. After the final determination or final establishment of the shape and/or size of the three-dimensional set of teeth in the CAD/CAM system, it is then possible for these data to be transmitted to a milling device that is known from the prior art, which processes the data and mills out the dental prosthesis.

The dental prosthesis produced in this way then advantageously corresponds to the patient's wishes and satisfies all medical and/or aesthetic aspects or requirements of the dental prosthesis.

According to an exemplary embodiment of this invention, the photograph of the set of teeth is selected from a tooth library, in which a large number of photographs of different sets of teeth are stored, the sets of teeth differing with regard to their surface textures, such as ridges on the teeth, tooth shapes, and/or tooth sizes.

The tooth library can, for example, be a printed catalog in which the patient can search for the optimal set of teeth in accordance with his or her wishes. The tooth library can also be in electronic form so that the different sets of teeth or the different photographs of the sets of teeth exist in the form of files and/or image files.

It is thus advantageously possible to offer the patient a wide variety of sets of teeth from which to select, thus making it advantageously possible to further improve patient satisfaction. It is naturally also possible to take into account medical aspects when selecting the set of teeth from the tooth library.

According to another exemplary embodiment of this invention, the photographs of the sets of teeth in the tooth library are photographs of real teeth. The term “real teeth” means that the photographs have been taken of the teeth of real people and/or patients. This advantageously improves the appearance of the set of teeth because the teeth of the set of teeth optimally fit one another.

According to another exemplary embodiment of this invention, the selection of the photograph of the set of teeth from the tooth library is made based on plaster or plastic molds of the photographed sets of teeth. These plaster or plastic molds exist in addition to the photographs of the sets of teeth.

The patient is thus able not only to look at the photograph, but also to inspect the entire three-dimensional appearance of the set of teeth. It is thus advantageously possible to optimally adapt the dental prosthesis to the patient's wishes.

According to another exemplary embodiment of this invention, the plaster or plastic molds are molds of real teeth that are identical to the corresponding photographed sets of teeth in the tooth library and the three-dimensional sets of teeth in the CAD/CAM system. This means that the plaster or plastic molds are molds of the same real teeth that are shown in the photographs of the sets of teeth. This enables a further improvement of the production of the dental prosthesis.

According to another exemplary embodiment of this invention, the plaster or plastic molds are divided into molds of male and female individuals. This makes it advantageously possible to take into account the different appearance of male and female teeth when selecting the set of teeth and thus when producing the dental prosthesis.

According to another exemplary embodiment of this invention, the patient's face is measured and the size and/or shape of the selected set of teeth in the photograph is determined depending on this facial measurement, and the teeth can be optimally positioned. The term “facial measurement” means that various points or lines are established, which can be taken into account in the production of the dental prosthesis. This enables an improved adaptation of the set of teeth to the conditions of the patient's mouth and/or face as well as from an aesthetic and functional standpoint.

According to another exemplary embodiment of this invention, the center of the patient's face is determined and the size and/or shape of the selected set of teeth in the photograph is/are determined based on the determined center of the face. In the context of this invention, the “center of the face” can be a point or a line.

The center of the face is thus a reference line or reference point that can be used to improve the dental prosthesis and/or the production method. The center of the face does not absolutely have to be the geometrical center point or geometrical symmetry line of the face but can instead also be a subjective reference line or subjective reference point determined by the dental technician.

According to another exemplary embodiment of this invention, in order to determine the center of the face, the patient's pupils are used as reference points. Since the patient's pupils play an important role for the symmetry of the face, it is thus advantageously possible to significantly improve the appearance of the set of teeth.

According to another exemplary embodiment of this invention, an optical device is used to determine the center of the face. Such a device is known, for example, from German Patent Reference DE 20 2004 016 058 U1, whose disclosure is hereby expressly and entirely included in this specification and in the disclosure content of the present application. With such a device, it is possible, for example, to mark the vertical center of the patient's face with improved precision. This therefore advantageously enables a further improvement of the production method.

According to another exemplary embodiment of this invention, a photograph of the face that is provided with a grid is used to determine the center of the face. This means that when the photograph of the face is taken, a grid, such as composed of vertical and horizontal lines, is held in front of the face so that grid lines or grid points in the photograph can be used as reference lines or reference points. It is thus possible to improve the production method.

The center of the face that is determined, such as with the device known from the German Patent Reference DE 20 2004 016 058 U1, can also be input into or transmitted to the CAD/CAM system so that an improved fit of the set of teeth can be achieved based on the possibility of also using the center of the face as a reference line in the CAD/CAM system.

Another subject of this invention relates to a dental prosthesis that has been produced using a method according to one of the preceding embodiments. Such a dental prosthesis is made of the conventional materials that are known from the prior art. With regard to the advantages of this dental prosthesis, we refer to the statements above regarding the method.

An exemplary embodiment of this invention is shown in the drawings and will be explained in greater detail in the description below.

### BRIEF DESCRIPTION OF THE DRAWINGS

FIG. 1 schematically shows a block circuit diagram that shows a sequence of the method according to this invention in conjunction with an exemplary embodiment; and

FIG. 2, FIG. 3, and FIG. 4 each shows an illustration of a patient's face.

### DETAILED DESCRIPTION OF THE INVENTION

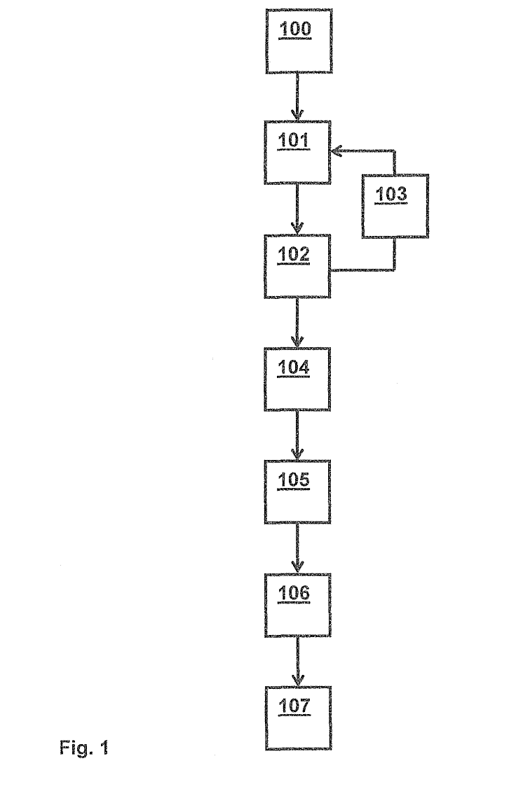

FIG. 1 schematically shows a block circuit diagram that shows the sequence of the method according to this invention in accordance with an exemplary embodiment.

Block 100 symbolizes the production of a digital photograph of the patient's face in a front view in which the patient's mouth is open and the set of the patient's teeth that are to be replaced is visible. This photograph shows, so to speak, the current state of the patient, with the current teeth. In this case, the current teeth can be visible, but it is also possible for only the processed tooth stumps to be visible.

It is important for the patient's mouth to be open and for the current set of teeth to be visible so that in the rest of the method, the replacement set of teeth can be inserted into the open mouth. The photograph of the face can also already be provided with a grid which can be used as reference points or reference lines. The center of the face is also advantageously determined in this method step. For example, the position of the pupils can thus be used. It is also possible, however, to use a conventional device for determining the center of the face, as is known from German Patent Reference DE 20 2004 016 058 U1.

Block 101 symbolizes the selection of a set of teeth that is to be used as a replacement for the patient's teeth. The patient can, for example, select the desired set of teeth from a tooth library, which is in the form of a catalog. Alternatively or in addition, the patient can also select the desired set of teeth from plaster or plastic molds.

Block 102 symbolizes the superimposition of the patient's teeth in the photograph of the patient's face by the digital photograph of the selected set of teeth by means of or with image processing software. To achieve this, for example, the photograph of the face and the photograph of the selected set of teeth are input into the image processing software. The photograph of the selected set of teeth can then be placed over the patient's current teeth in the image processing software.

The patient can now see how the selected set of teeth would look in his or her mouth and can decide whether or not he or she likes the appearance of the set of teeth. If the patient is not satisfied, then the method goes back to block 101 so that another set of teeth is selected, for example from the tooth library. This return is symbolized by block 103.

When the patient is satisfied with the appearance of the set of teeth, then in block 104, the size and/or shape of the selected set of teeth is adapted to the patient's mouth and/or face in the image processing software. This adaptation is helpful, for example, if the center of the patient's face has been determined and/or if the patient's face has been provided with a grid.

It is then possible, for example, to adapt the size and shape of individual teeth. It is also possible to change the positions of the teeth. In this adaptation of the set of teeth, it is also possible to take into account the patient's wishes. If it should turn out at this stage that the patient does not in fact like the set of teeth, then it is also possible to go from this method step back to block 101 and select a new set of teeth.

In this method step or block 104, it is also possible to take into account medical aspects and the dental technician can introduce additional aesthetic aspects. It is advantageously possible at any time to take into account the future appearance of the set of teeth in the patient's mouth so that it is advantageously possible to carry out an optimal adaptation of the set of teeth.

After the method step symbolized by block 104 has been completed, the ascertained data are transmitted to a conventional CAD/CAM system. This transmission is symbolized by block 105 and can occur automatically, but can also be carried out manually by the dental technician. After the data have been transmitted to the CAD/CAM system, the three-dimensional data set that corresponds to the selected set of teeth is called up in the CAD/CAM system.

For each set of teeth that can be selected, a corresponding data set can be stored in the CAD/CAM system. This data set is selected and processed as a function of the data established in the image processing software. This processing is symbolized by block 106. The desired size and/or shape is/are thus transmitted to the CAD/CAM system so that the depiction of the set of teeth and/or the size and shape of the set of teeth correspond to the photographed depiction in the CAD/CAM system.

In this method step, the dental technician can naturally also continue processing the size and/or shape of the set of teeth in the CAD/CAM system. In the final method step, the processing data finally established in the CAD/CAM system are transmitted to a conventional milling device in order to finally produce the desired dental prosthesis. This method step is symbolized by block 107. The manufacturing method has therefore been completed and the produced dental prosthesis can be sent to the dentist for insertion into the patient's jaw.

Another advantage of the method according to this invention is that the planning and design of the dental prosthesis, both of a possible wax-up or temporary appliance and of the definitive dental prosthesis, are established at the beginning of the method and are then maintained throughout the entire process despite the existence of significant individual/manual method steps.

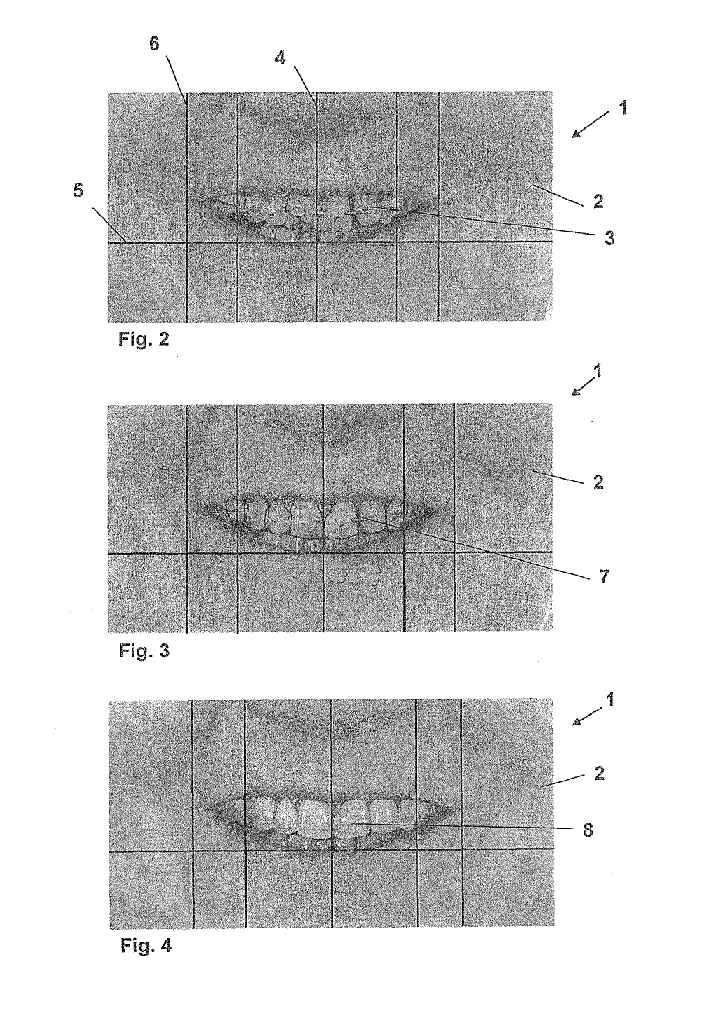

FIG. 2 shows an image 1 of a patient's face 2. In this case, the image is a digital photograph. FIG. 2 shows a front view of the patient's face 2 in which the patient's teeth 3 are still in the situation with the teeth that are damaged and/or are to be replaced. In this case, the teeth 3 of the upper jaw are to be replaced. The teeth of the lower jaw will remain unchanged.

In order to measure the face 2, the mouth, and the teeth 3, reference lines 4, 5, have been drawn into the image. In this case, there is one horizontal reference line 5 and there are five vertical reference lines 4, 6. The horizontal reference line 5 extends approximately through the point at which the lowest part of the upper jaw meets the lower lip.

The vertical reference line all the way to the left extends through the patient's pupil. For the sake of simplifying the illustration, the pupils are not shown in this figure. The second vertical reference line from the left extends approximately through the patient's cuspid that is to be replaced. The middle horizontal and vertical reference line 4 corresponds approximately to the center of the patient's face 4. The second vertical reference line from the right extends approximately through the patient's other cuspid that is to be replaced. The vertical reference line all the way to the right extends approximately through the patient's other pupil. FIG. 2 shows the initial state of the patient and/or the patient's teeth before the production of the dental prosthesis.

Like FIG. 3, FIG. 2 shows a front view of the patient's face 2 and/or mouth in a digital photograph. The photograph contains the same reference lines from FIG. 2 that have been described above. In FIG. 3, outlines 7 are visible, which correspond to the new teeth or outlines 7 of the selected set of teeth. The selected set of teeth and/or the selected teeth are shown superimposed over the original teeth.

FIG. 3 shows the differences between the original situation and the situation with the selected set of teeth. The size and shape of the teeth have been adapted according to this invention so that the desired medical and/or aesthetic aspects have been satisfied and the teeth fit in the patient's mouth in a suitable way. As an additional step, it would be possible, for example, to measure the outlines 7 of the new teeth. These measurement results could then be input into the CAD/CAM system so that the determined measurement values can be converted into the three-dimensional image of the CAD/CAM systems.

Like FIGS. 2 and 3, FIG. 4 shows a front view of the patient's face 2. In FIG. 4, the selected teeth or the selected set of teeth 8 are/is inserted into the patient's mouth, with the original teeth 3 being superimposed by the selected set of teeth 8. At this stage of the method, the patient can decide whether or not he or she agrees with the future situation of the teeth or set of teeth. If not, then another set of teeth can be selected and the method begins again.

1. A method for producing a dental prosthesis, including the steps of:
producing an image (1) of a front view of a patient's face (2) in which a patient's mouth is open and a set of the patient's teeth (3) being replaced is visible;
superimposing the patient's teeth (3) in the image (1) of the patient's face (2) by a digital photograph of a selected set of teeth (8) with image processing software;
adapting a size and/or shape of the selected set of teeth (8) in the photograph of the patient's mouth and/or face (2) with the image processing software;
transmitting the obtained size and/or shape of the selected set of teeth (8) to a CAD/CAM system;
selecting in the CAD/CAM system a three-dimensional set of teeth that corresponds to the selected set of teeth (8);
adapting the three-dimensional set of teeth in the CAD/CAM system based on the size and/or shape of the selected set of teeth determined in the photograph; and
milling the dental prosthesis out of a block of material based on the size and/or shape of the adapted three-dimensional set of teeth determined in the CAD/CAM system.

2. The method according to claim 1, wherein the photograph of the set of teeth (8) is selected from a tooth library of a large number of stored photographs of different sets of teeth, and the sets of teeth differing with regard to their tooth shapes and/or tooth sizes.

3. The method according to claim 2, wherein the photographs of the sets of teeth (8) in the tooth library are photographs of real teeth.

4. The method according to claim 3, wherein the selection of the photograph of the set of teeth (8) from the tooth library is carried out based on plaster or plastic molds of the photographed sets of teeth.

5. The method according to claim 4, wherein the plaster or plastic molds are molds of real teeth that are identical to the corresponding photographed sets of teeth (8) in the tooth library and the three-dimensional sets of teeth in the CAD/CAM system.

6. The method according to claim 5, wherein the plaster or plastic molds are divided into molds of male and female individuals.

7. The method according to claim 6, wherein the patient's face (2) is measured and the size and/or shape of the selected set of teeth (8) in the photograph is/are established based on a facial measurement.

8. The method according to claim 7, wherein the center of the patient's face (4) is determined and the size and/or shape of the selected set of teeth (8) in the photograph is/are established based on the determined center of the face (4).

9. The method according to claim 8, wherein the patient's pupils are used as reference points for determining the center of the face (4).

10. The method according to claim 9, wherein an optical device is used to determine the center of the face (4).

11. The method according to claim 10, wherein a photograph of the face (2) that is provided with a grid (5, 6) is used to determine the center of the face (4).

12. A dental prosthesis, produced using the method according to claim 11.

13. The method according to claim 2, wherein the selection of the photograph of the set of teeth (8) from the tooth library is carried out based on plaster or plastic molds of the photographed sets of teeth.

14. The method according to claim 13, wherein the plaster or plastic molds are molds of real teeth that are identical to the corresponding photographed sets of teeth (8) in the tooth library and the three-dimensional sets of teeth in the CAD/CAM system.

15. The method according to claim 1, wherein the patient's face (2) is measured and the size and/or shape of the selected set of teeth (8) in the photograph is/are established based on a facial measurement.

16. The method according to claim 15, wherein the center of the patient's face (4) is determined and the size and/or shape of the selected set of teeth (8) in the photograph is/are established based on the determined center of the face (4).

17. The method according to claim 8, wherein an optical device is used to determine the center of the face (4).

18. The method according to claim 9, wherein a photograph of the face (2) that is provided with a grid (5, 6) is used to determine the center of the face (4).

19. The method according to claim 8, wherein a photograph of the face (2) that is provided with a grid (5, 6) is used to determine the center of the face (4).

20. A dental prosthesis, produced using the method according to claim 1.
